(19) United States
(12) Patent Application Publication
(10) Pub. No.: US 2022/0088322 A1
(43) Pub. Date: Mar. 24, 2022

(54) LASER VAPORIZER

(71) Applicant: IFEDAYO OLUSEYI ADEDAYO OLADAPO, Katy, TX (US)

(72) Inventor: IFEDAYO OLUSEYI ADEDAYO OLADAPO, Katy, TX (US)

(21) Appl. No.: 17/025,534

(22) Filed: Sep. 18, 2020

(51) Int. Cl. A61M 11/04; G02B 26/10; B23K 26/046

(52) U.S. Cl. CPC A61M 11/042; A61M 15/0086; B23K 26/046; G02B 26/105

(57) ABSTRACT

A laser vaporizer includes a laser, a heating vial, and an exit port. The laser heats a target substance to a vaporization temperature in a vaporization chamber. The vaporization chamber is made of glass or other inert material. The laser vaporizer is configured to safely deliver vaporized medicines, plant material and extracts without introducing further contaminants to the vapor so as to prevent the leaching of elements into the vapor. The vaporized target substance is passed through the exit port for inhalation.

### BACKGROUND OF THE INVENTION

### 1. Field of the Invention

The present invention relates to Vaporizers for e-liquids and other target substances and in particular to the use of lasers to heat the target substance to vaporization temperature in a vaporization chamber made of glass or other inert material.

### 2. Description of Related Art

Vaporizers are devices used to vaporize substances for inhalation. Plants such as tobacco, cannabis and other herbs or extract oils and isolates which may be diluted in glycerol emulsions. The latter emulsions are referred to as e-liquids. Some Variants of vaporizers use an undiluted extract, dry plant material or a solid waxy plant extract as a target.

Existing e-liquid vaporizers have come under scrutiny for potential adverse, effects on pulmonary health. Some of these problems such as lipid pneumonia are related to the make-up of the e-liquid. More complex case, histories of cellular, Pulmonary and cardiovascular disease indicate other sources of harmful substances in the vapor. Recent analysis of vapors coming from tank style vaporizers show heavy metal content including, chromium, copper lead, nickel and zinc in excess of proposed permissible exposure limits for occupational hazards. These metals likely originate from components of the atomizer unit which consists of a heating filament usually nichrome, alloy wire, tin solder joints, brass clamps, insulating sheaths and Wick. Vaporizers with fewer metal parts also have less metal in the resulting vapor.

Vaporizers have a wide variety of applications ranging from recreational to medical to culinary. A vaporizer heats a material for vaporization allowing precise thermal control to target evaporation temperatures of specific compounds. The resulting vapor contains almost no particulate matter or tar, nor does it contain byproducts of the combustion process such as carbon monoxide. The vapor may be inhaled or otherwise used directly or collected in a bag prior to use. Vaporizers typically heat the target substance in a metal or glass enclosure. A resistive electrical heat source is used in most devices although some vaporizers use combustion as a heat source. In high powered devices the metals in the resistive heating element and other components may leach into the e-liquid, the vapor and the user's lungs attenuating risks for a range of health complications.

Examples of prior art include embodiments found in FIGS. 1-3. In these embodiments, the wick is made of an absorbent material which distributes the target substance 109 evenly along the length of the heating filament which surrounds it. The perforated screen 113 forms a protective but permeable layer around the heating filament 111 and wick 112. The perforated screen 113, heating filament 111 and wick 112 are housed within the heating vial 102, where they sit immersed in the target substance 109. The heating vial 102 along with the heating filament 111, wick 112 and perforated screen 113 within it, may be a sealed replaceable cartridge or have a removable heating vial lid 101 to allow for refilling.

The target substance 109 is vaporized by passing electrical current through the resistive heating filament 111. With the exception of the heating mechanism the principles of operation are similar. The control panel 104 delivers electrical power from the battery 106 via the power supply 108 which also handles charging via a USB port 115. The control panel 104 also has a digital display 105 for user interface controls. The target substance 109 vapor is removed via a vapor outlet 103 and pressure is equalized through an air valve 114.

Although strides have been made, shortcomings remain. What is needed is a vaporizer that can safely deliver vaporized medicines, plant material and extracts without introducing further contaminants to the vapor.

### BRIEF SUMMARY OF THE INVENTION

It is an object of the present application to provide a vaporizer that can deliver vapors of medicines. e-liquids and plant extracts in a manner that avoids the introduction of contaminants from the device itself.

It is an object of this invention to provide a vaporizer that uses a non-contact method of heating that can be applied to liquids such as e-liquids or pure extracts and solids such as powdered crystals. dry plant matter or waxy plant extracts.

The vaporizer will be able to heat a target substance to vaporization temperature and sustain this temperature without heating other materials that may leach into the vapor. In order to overcome the challenge of vaporizing the target substance without ingress of metals into the vapor, the vaporizer uses a laser to vaporize the target substance in a vaporization chamber made of glass or other inert material. It is desired that the vaporization chamber is inert at the temperatures required for operation. The laser will be positioned outside the vaporization chamber. The laser beam will be redirected using mirrors and focused through an aperture at the end of the chamber.

Ultimately the invention may take many embodiments. In these ways, the present invention overcomes the disadvantages inherent in the prior art. The more important features have thus been outlined in order that the more detailed description that follows may be better understood and to ensure that the present contribution to the art is appreciated. Additional features will be described hereinafter and will form the subject matter of the claims that follow.

Many objects of the present application will appear from the following description and appended claims, reference being made to the accompanying drawings forming a part of this specification wherein like reference characters designate corresponding parts in the several views.

Before explaining at least one embodiment of the present invention in detail, it is to be understood that the embodiments are not limited in its application to the details of construction and the arrangements of the components set forth in the following description or illustrated in the drawings. The embodiments are capable of being practiced and carried out in various ways. Also it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the various purposes of the present design. It is important, therefore, that the claims be regarded as including such equivalent constructions insofar as they do not depart from the spirit and scope of the present application.

### BRIEF DESCRIPTION OF THE DRAWINGS

The novel features believed characteristic of the application are set forth in the appended claims. However, the application itself, as well as a preferred mode of use, and further objectives and advantages thereof, will best be understood by reference to the following detailed description when read in conjunction with the accompanying drawings, wherein:

FIG. 1 is a perspective cross section view of a prior art vaporizer.

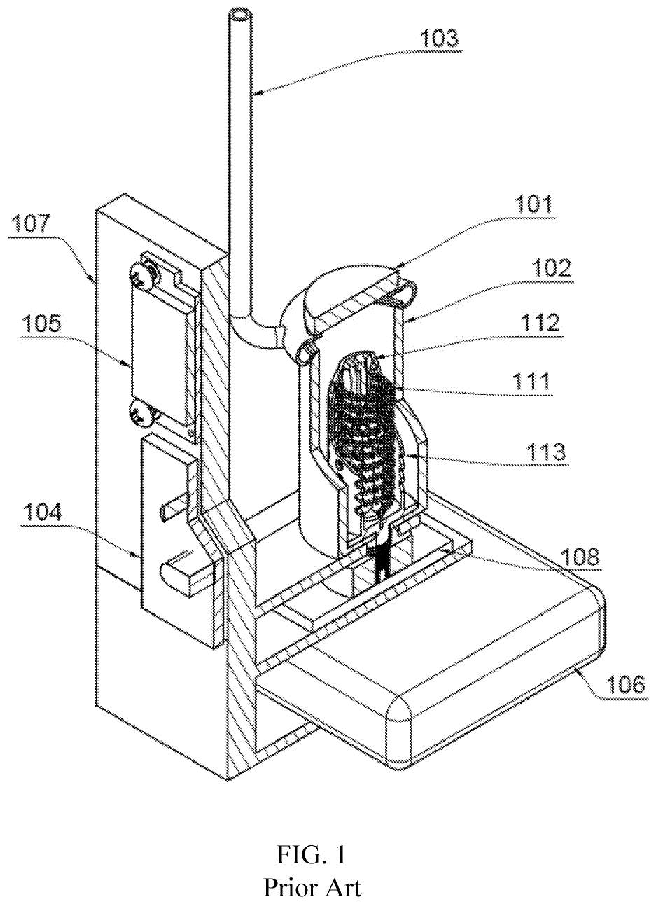

FIG. 2 is a detailed view of the prior art vaporizer of FIG. 1.

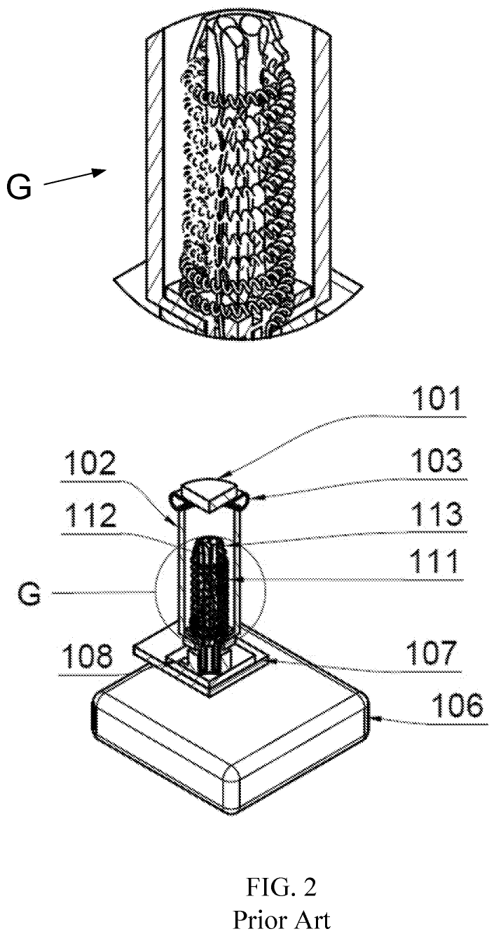

FIG. 3 is an exploded view of the prior art vaporizer of FIG. 1.

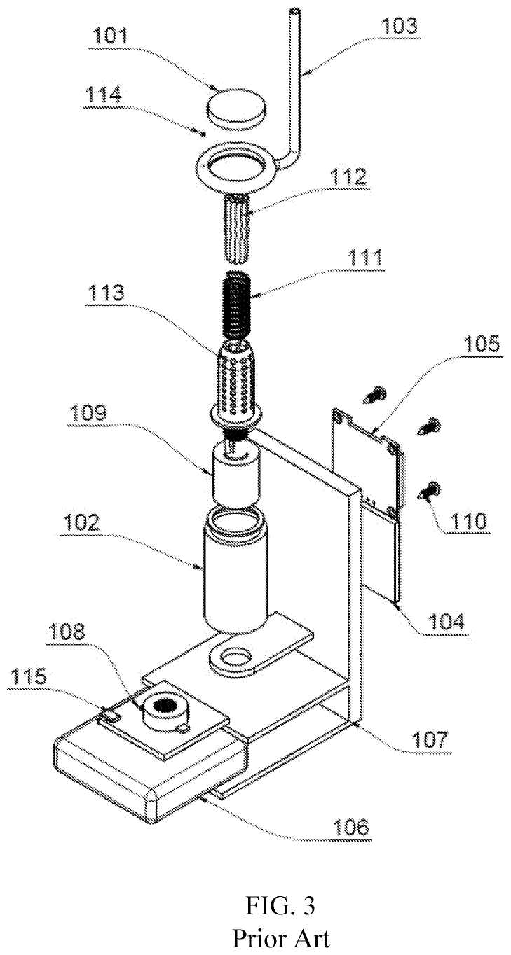

FIG. 4 is a rear view of a laser vaporizer according to an embodiment of the present application.

FIG. 5 is a rear cross section view of the laser vaporizer of FIG. 4.

FIG. 6 is a left view of the laser vaporizer of FIG. 4.

FIG. 7 is a left cross section view of the laser vaporizer of FIG. 6.

FIG. 8 is a front view of the laser vaporizer of FIG. 4.

FIG. 9 is a front cross section view of the laser vaporizer of FIG. 8.

FIG. 10 is a perspective view of the laser vaporizer of FIG. 4.

FIG. 11 is a cross section view of the laser vaporizer of FIG. 10.

FIG. 12 is an exploded view of the laser vaporizer of FIG. 10.

While the embodiments and method of the present application is susceptible to various modifications and alternative forms, specific embodiments thereof have been shown by way of example in the drawings and are herein described in detail. It should be understood, however, that the description herein of specific embodiments is not intended to limit the application to the particular embodiment disclosed, but on the contrary, the intention is to cover all modifications, equivalents, and alternatives falling within the spirit and scope of the process of the present application as defined by the appended claims.

### DETAILED DESCRIPTION OF THE INVENTION

Illustrative embodiments of the preferred embodiment are described below. In the interest of clarity, not all features of an actual implementation are described in this specification. It will of course be appreciated that in the development of any such actual embodiment, numerous implementation-specific decisions must be made to achieve the developer's specific goals, such as compliance with system-related and business-related constraints, which will vary from one implementation to another. Moreover, it will be appreciated that such a development effort might be complex and time-consuming but would nevertheless be a routine undertaking for those of ordinary skill in the art having the benefit of this disclosure.

In the specification, reference may be made to the spatial relationships between various components and to the spatial orientation of various aspects of components as the devices are depicted in the attached drawings. However, as will be recognized by those skilled in the art after a complete reading of the present application, the devices, members, apparatuses, etc. described herein may be positioned in any desired orientation. Thus, the use of terms to describe a spatial relationship between various components or to describe the spatial orientation of aspects of such components should be understood to describe a relative relationship between the components or a spatial orientation of aspects of such components, respectively, as the embodiments described herein may be oriented in any desired direction.

The embodiments and method in accordance with the present application overcomes one or more of the above-discussed problems commonly associated with the prior art discussed previously. In particular, the laser vaporizer of the present application uses lasers to heat a target substance to vaporization temperature in a vaporization chamber. The vaporization chamber is made of glass or other inert material. The laser vaporizer is configured to safely deliver vaporized medicines, plant material and extracts without introducing further contaminants to the vapor. These and other unique features are discussed below and illustrated in the accompanying drawings.

The embodiments and method will be understood, both as to its structure and operation, from the accompanying drawings, taken in conjunction with the accompanying description. Several embodiments of the assembly may be presented herein. It should be understood that various components, parts, and features of the different embodiments may be combined together and/or interchanged with one another, all of which are within the scope of the present application, even though not all variations and particular embodiments are shown in the drawings. It should also be understood that the mixing and matching of features, elements, and/or functions between various embodiments is expressly contemplated herein so that one of ordinary skill in the art would appreciate from this disclosure that the features, elements, and/or functions of one embodiment may be incorporated into another embodiment as appropriate, unless otherwise described.

Referring now to the Figures wherein like reference characters identify corresponding or similar elements in form and function throughout the several views. The following Figures describe embodiments of the present application and its associated features. With reference now to the Figures, embodiments of the present application are herein described. It should be noted that the articles “a”, “an”, and “the”, as used in this specification, include plural referents unless the content clearly dictates otherwise.

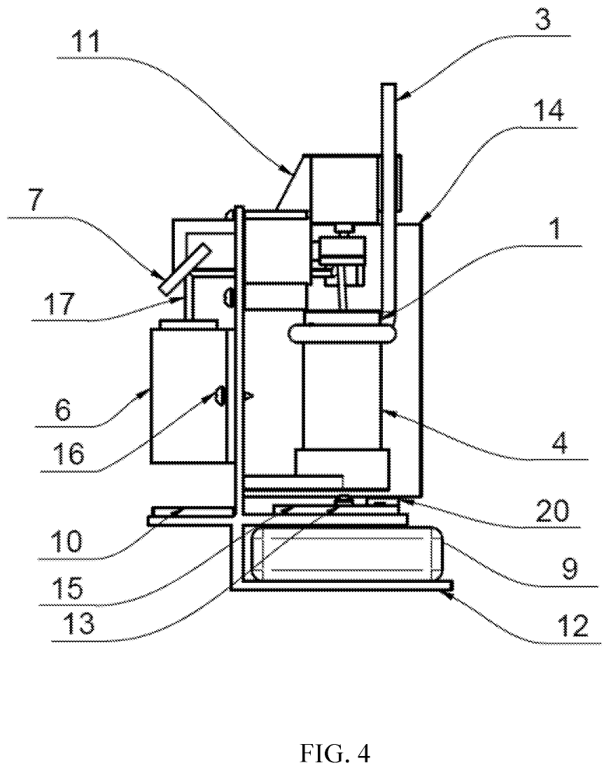

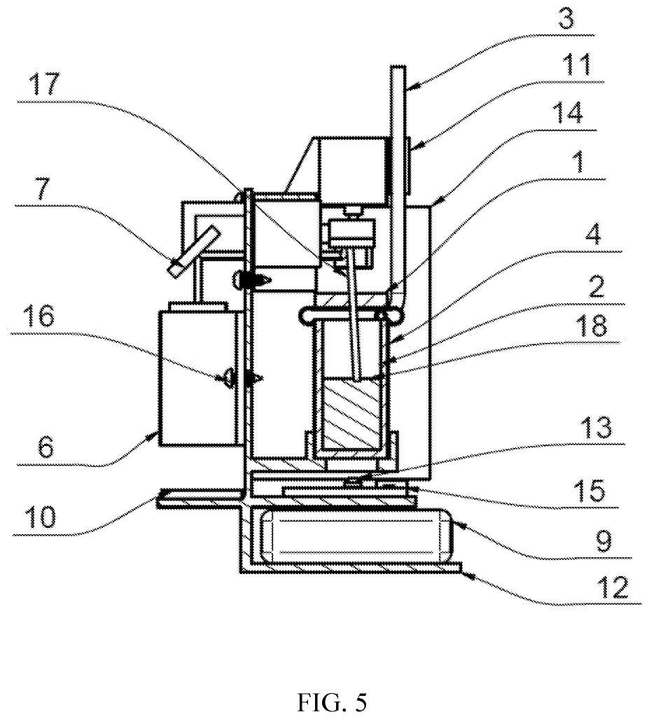

Referring now to FIGS. 4-12 in the drawings, an exemplary arrangement of a laser vaporizer is disclosed. FIGS. 4 and 5 show a rear view and a rear view cross section respectively. A laser diode 6 is affixed upon a support 12. The emitted laser beam 17 is focused using a set of lenses enclosed in the housing of the laser diode 6 and then reflected first through a fixed 90 degree mirror 7. Subsequently it is then redirected off a galvanometer 11 and a pair of motorized scanning mirrors. The path of the laser beam 17 continues by passing through a laser aperture window 1 into the heating vial 2 containing the target substance 18 to be vaporized.

A laser driver 10 controls the laser diode 6 and galvanometer 11 to precisely target the laser beam 17 as it evenly sweeps the surface of the target substance 18. The laser driver 10 allows two operating modes. A first operating mode is for heating the target substance 18 and a secondary operating mode is for measuring the quantity of remaining target substance 18 in the heating vial 2. A control algorithm, such as a proportional integral differential controller located in the laser driver 10, precisely controls the temperature of the target substance 18 during operation using any of measured inputs from a pyrometer 13, inputs from an optical thermometer mounted underneath the heating vial 2 or modeled inputs from a predictive temperature model as with devices that are not equipped with a pyrometer 13.

The laser beam 17 vaporizes the target substance in a controlled manner. It is pulsed to steadily maintain the desired temperature at the surface of the target substance 18. As the target substance 18 is vaporized, the vapor rises up through heating vial 2 and can be extracted by applying a suction to the vapor outlet 3. Thereafter the operator may inhale or collect the vapor as required.

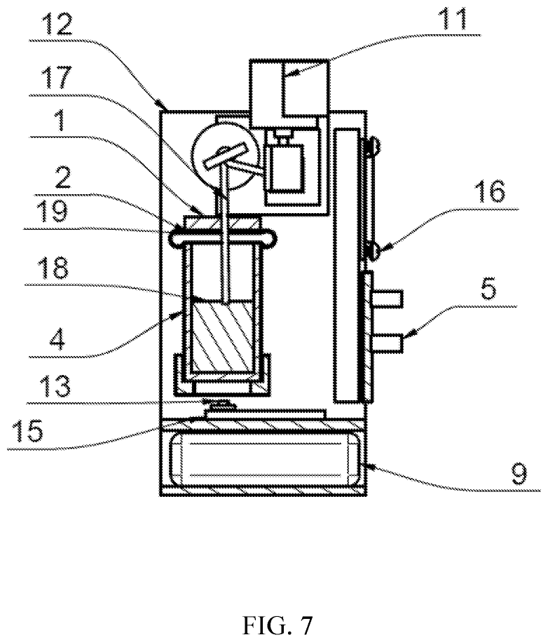

To allow for displacement of air upon the application of suction to the vapor outlet 3, there is a small air valve 19 opposite the vapor outlet 3 as seen in FIGS. 7, 10, and 11.

Furthermore, the outside of the heating vial 2 is lined with a laser protective screen 4 to diffuse, or in general, protect operators and bystanders from harmful laser radiation.

The laser aperture window 1 may be removable so that the heating vial 2 can be refilled or the heating vial 2 may be sealed as a replaceable cartridge.

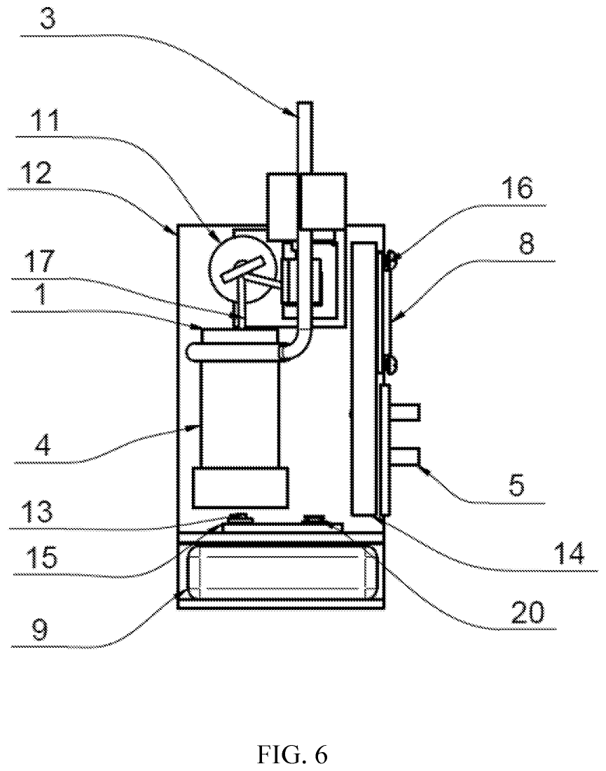

Referring now also to FIGS. 6 and 7. Here the same arrangement is depicted from the side, as compared to FIGS. 4 and 5. There is a control panel 5 that provides an input/output interface for the operator with control buttons and a digital display 8. The galvanometer 11 is regulated using modeled or measured inputs detailing the level of remaining target substance 18 and the orientation of the device obtained by a gyroscope mounted on the control panel 5 circuit.

Electrical power is provided by a rechargeable battery 9. A power supply 15 circuit provides the various operating voltages required by the laser driver 10, control panel 5 and galvanometer 11, respectively. The power supply 15 also handles battery management and charging via a USB Port 20. Various forms of power supplies may be used. It is understood that the power supply may be a battery 9 or hardwired electrical connection. A power management board may be used to regulate delivery of voltage to any of the operative components in the laser vaporizer or any of its electrical subsystems.

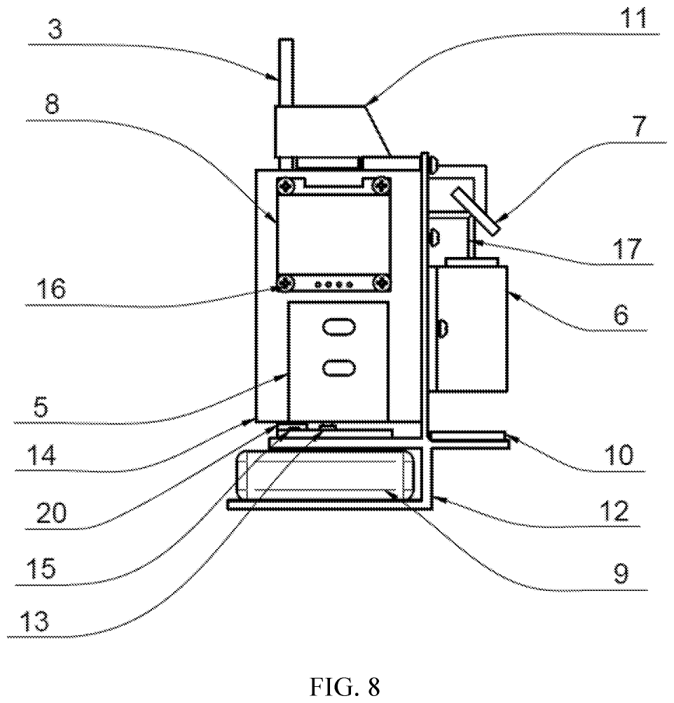

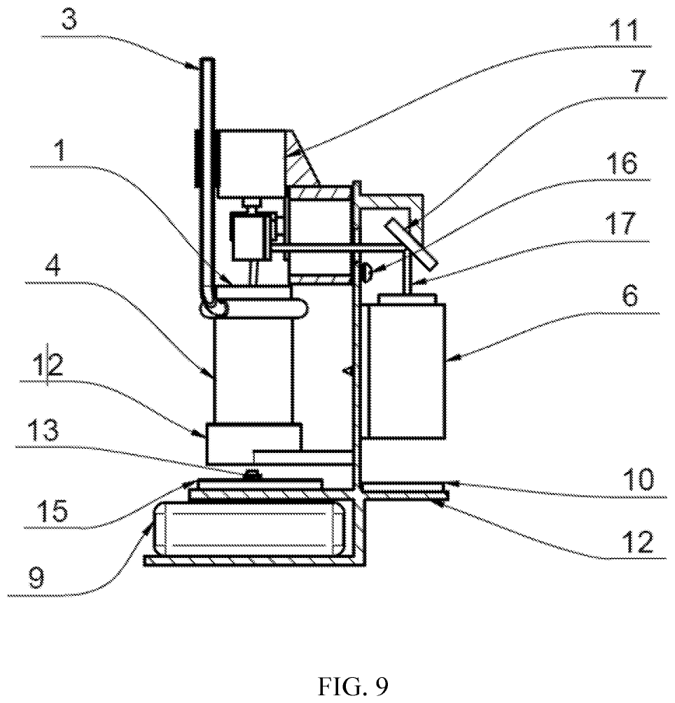

Referring now also to FIGS. 8 and 9 in the drawings. FIG. 8 shows the front of the vaporizer with the control panel 5 in plain view. The control panel 5 is fastened to it's own support 14 with a fastener such as a screw 16. Likewise the laser diode 6 is fastened to it's support 12. The display provides the operator with information about the temperature, amount of target substance 18 vaporized, and quantity of remaining target substance 18 in the heating vial 2. FIG. 9 shows a cross section revealing the path of the laser beam 17 through the fixed 90 degree mirror 7 and the motorized mirrors of the galvanometer 11.

Optionally the addition of a linear image sensor and corresponding optics may be used. These may add some spectrometry functionality and inform the operator of the chemical composition of the vapor. The temperature control regulator may further be enhanced by augmenting or replacing the pyrometer 13 with a thermal imaging camera.

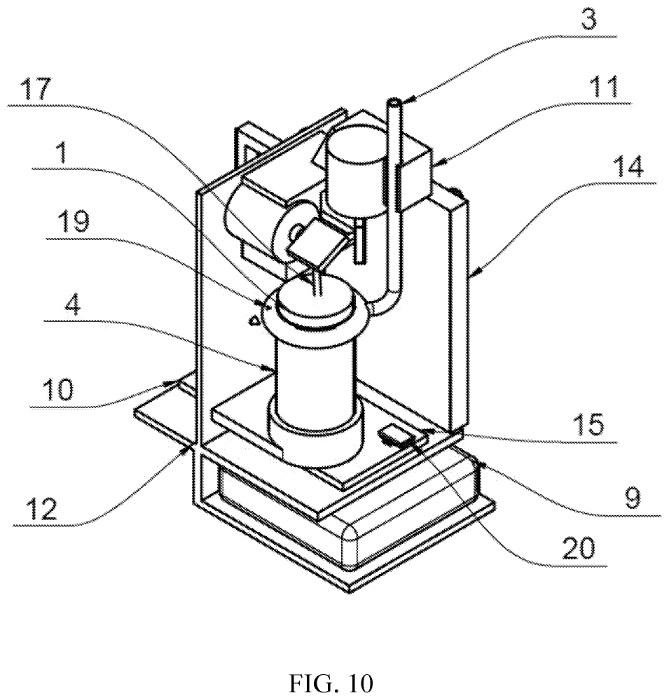

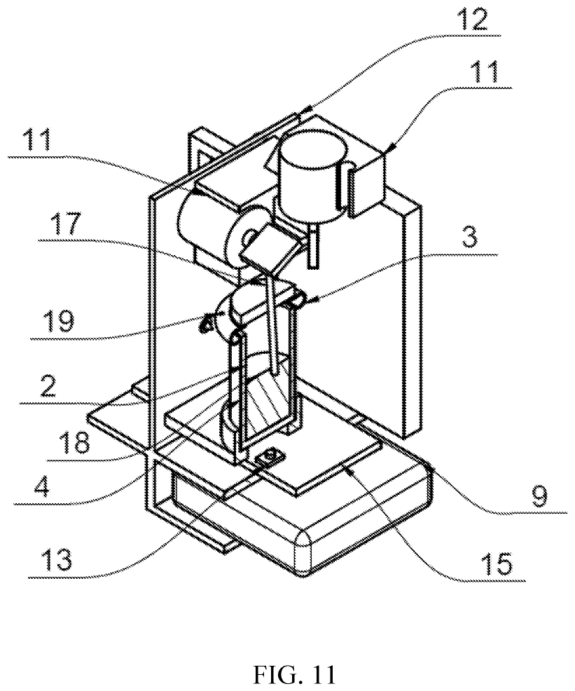

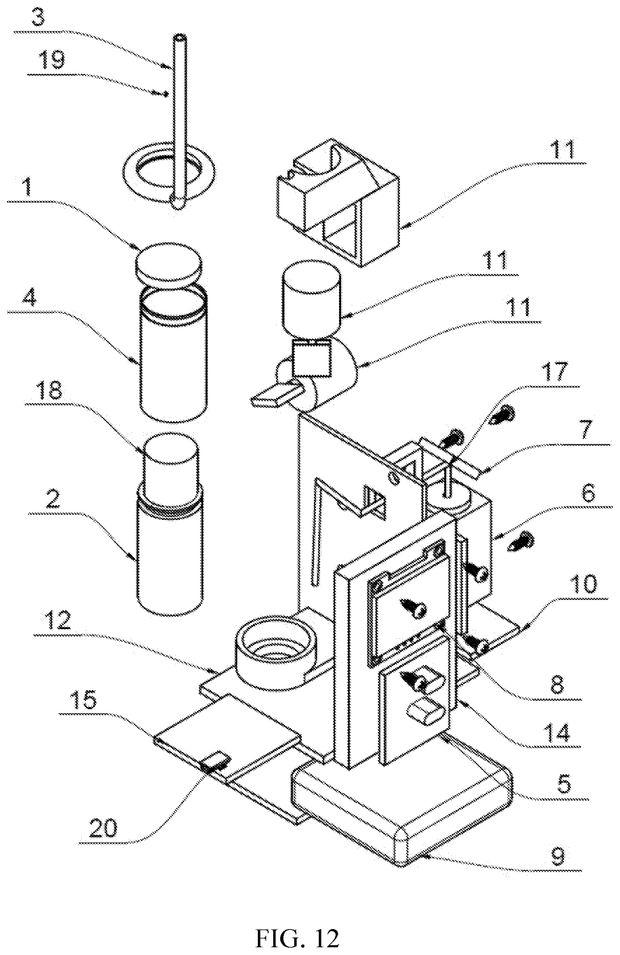

Referring now also to FIGS. 10-12 in the drawings. FIGS. 10 and 11 show a perspective view of the device. FIG. 12 shows an exploded view revealing almost all the annotated components of this preferred embodiment.

It is understood that the device of the present application may be sized in accordance with design constraints. This may lead to a hand-held version or an embodiment larger such that it is affixed to a surface.

The particular embodiments disclosed above are illustrative only, as the application may be modified and practiced in different but equivalent manners apparent to those skilled in the art having the benefit of the teachings herein. It is therefore evident that the particular embodiments disclosed above may be altered or modified, and all such variations are considered within the scope and spirit of the application. Accordingly, the protection sought herein is as set forth in the description. It is apparent that an application with significant advantages has been described and illustrated. Although the present application is shown in a limited number of forms, it is not limited to just these forms, but is amenable to various changes and modifications without departing from the spirit thereof.

What is claimed is:

1. A vaporizer used to vaporize a substance for inhalation, comprising:
a laser for heating a target substance;
a heating vial; and
an exit through which the target substance may be collected;
wherein the heated target substance passes through the exit as a vapor.

2. The vaporizer of claim 1, further comprising a thermal imaging camera to measure the temperature inside the heating vial.

3. The vaporizer of claim 1, further comprising a gyroscope configured to determine the orientation of the target substance.

4. The vaporizer of claim 3, further comprising a galvanometer;
wherein the gyroscope is configured to regulate the operation of the laser focus and galvanometer.

5. The vaporizer of claim 1, further comprising a linear image sensor configured to provide chemical data related to the target substance.

6. The vaporizer of claim 5, wherein the linear image sensor provides spectrometric analysis of the vapor.

7. The vaporizer of claim 1, wherein the laser is configured to operate at different intensities, the intensity being adjustable.

8. The vaporizer of claim 7, wherein adjustment of the intensity of the laser to a lesser level permits the laser to measure the quantity and orientation of the target substance.

9. The vaporizer of claim 1, further comprising one or more mirrors configured to regulate the path of the laser.

10. The vaporizer of claim 9, wherein the one or more mirrors may have a fixed orientation or an adjustable orientation.

11. The vaporizer of claim 1, further comprising a user interface having a display and a control input.

12. The vaporizer of claim 1, further comprising a laser driver configured to target the laser.

13. The vaporizer of claim 1, further comprising a power system including:
a rechargeable battery;
a power management board for regulating delivery of operating voltage to the laser and electrical subsystems; and
a connector for charging the battery.

14. The vaporizer of claim 13, wherein the connector is a diagnostic interface.

15. The vaporizer of claim 1, further comprising a protective screen coupled to the heating vial to diffuse laser radiation.

16. The vaporizer of claim 1, further comprising a galvanometer configured to control the path of the laser.

17. The vaporizer of claim 1, further comprising a pyrometer to measure the temperature inside the heating vial.
